(19) United States
(12) Patent Application Publication
(10) Pub. No.: US 2011/0196267 A1
(43) Pub. Date: Aug. 11, 2011

(54) ULTRASOUND NEUROMODULATION OF THE OCCIPUT

(76) Inventor: David J. Mishelevich, Playa del Rey, CA (US)

(21) Appl. No.: 13/021,785

(22) Filed: Feb. 7, 2011

Related U.S. Application Data
(60) Provisional application No. 61/302,160, filed on Feb. 7, 2010.

(51) Int. Cl. A61N 7/00

(52) U.S. Cl. 601/2

(57) ABSTRACT

Disclosed are methods and systems for non-invasive neuromodulation of the occipital nerves using ultrasound transducers to treat migraine and cluster headaches in their multiple variations as well other pain and tension conditions. Treatment may be unilateral or bilateral.

### CROSS REFERENCE TO RELATED APPLICATIONS

This patent application claims priority to provisional patent applications Application No. 61/302,160, filed Feb. 7, 2010, entitled “ULTRASOUND NEUROMODULATION OF THE OCCIPUT.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation of the occipital nerve and related neural structures.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. One or a plurality of neural elements can be neuromodulated.

Potential application of ultrasonic therapy of deep-brain structures has been covered previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). It was noted that monophasic ultrasound pulses are more effective than biphasic ones.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Patent applications have been filed addressing neuromodulation of deep-brain targets (Bystritsky, “Methods for modifying electrical currents in neuronal circuits,” U.S. Pat. No. 7,283,861, Oct. 16, 2007 and Deisseroth, K. and M. B. Schneider, “Device and method for non-invasive neuromodulation,” U.S. patent application Ser. No. 12/263,026 published as US 2009/0112133 A1, Apr. 30, 2009).

Transcranial Magnetic Stimulation (TMS) has been successfully used in occipital nerve stimulation for migraine headache and other headaches. For example in Mohammed et al. (Mohammad, Y. M., Kothari, R., Hughes, G., Nkrumah, M., Fischell, S., Fischell, R. F., Schweiger, J., and P. Ruppel, “Transcranial Magnetic Stimulation (TMS) relieves migraine headache,” Abstract, American Headache Society Meeting 2006), two TMS pulses were delivered, 30 seconds apart. The treatment was well tolerated and there was a tendency to reduce pain at two hours as well as nausea and cognitive function in the double blind, placebo controlled study. In a single pulse TMS study Lipton et al. (Lipton, R. B., Dodick, D. W., Goadsby, P. J., Saper, J. R., Silberstein, S. D., Aurora S. K., Mohammad, Y. M.; Ruppel, P. L., and R. E. Fischell, “Transcranial Magnetic Stimulation (TMS) Using a Portable Device is Effective for the Acute Treatment of Migraine with Aura: Results of a Double Blind, Sham Controlled, Randomized Study,” Abstract, American Headache Society Meeting June 2008) in which there was also relief at two hours. In another study Clarke et al. (Clarke, B. A., Upton, A. R. M., Kamath, M. V., Al-Harbi, T., and C. M. J. Castellanos. “Transcranial magnetic stimulation for migraine: clinical effects,” Headache Pain, 7:341-346, 2006) involving two-pulse stimulation of the autonomic nervous system to treat migraine headache, if patient had aura, improvement was typically immediate majority of patients got relief with no adverse side effects.

Deep-brain stimulation (DBS) of the occipital nerves has also been used to treat headache and other maladies. For example, Burns et al. studied cluster headaches (Burns, B., Watkins, L., and P. Goadsby, “Treatment of medically intractable cluster headache by occipital nerve stimulation: long-term follow-up of eight patients,” The Lancet, Volume 369, Issue 9567, Pages 1099-1106, 31 March 2007). Seven of the patients were bilaterally stimulated and one unilaterally stimulated and six of the eight patients reported meaning responses with improvement in both frequency and severity of attacks.

Autonomic stimulation to positively impact intracranial structures such as the Vagal Nerve Stimulation (VNS) is used successfully in clinical practice (e.g., George, M., Sackheim, A J, Rush, et al., “Vagus Nerve Stimulation: A New Tool for Brain Research and Therapy,” Biological Psychiatry, 47, 287-295, 2000).

Electrical stimulation, including autonomic nervous system stimulation, has been associated with treatment of headaches and associated symptoms such as nausea and vomiting. A variety of non-invasive treatments have been used for headache treatment such as medication, diet, trigger avoidance, acupuncture, anesthetic agents, biofeedback, and physical therapy. Invasive treatments have been used as well such as ganglion resection, ganglion block, radiosurgery, and cryotherapy. Electrical stimulation has been applied by implanted electrodes or implanted stimulator. A stimulator can be set to deliver a predetermined pattern of stimulation, or the patient may control the amplitude, pulse width, and frequency using a remote-control device.

Such stimulation has also been associated with the treatment of a number of other conditions including neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

Many of the sensory and motor nerves of the neck are contained in C2 and C3, including the Greater Occipital Nerve (GON). NeurologyReviews.com (Vol. 16, Num. 10, October 2008) reviewed considerations of stimulation of the occipital nerve in treatments of headaches such as migraine, cluster, and hemicrania continua. Blocks of the occipital nerve have had success in treatment of headache in its various forms. An important aspect is that positive effect of the treatment outlasts the impact of the neural block. This indicates that there is some longer-term neuromodulation. Such blocks, while effective in a majority of cases, are not always predictive of whether longer-term occipital nerve electrical stimulation will be successful. In some cases, there is a delayed effect (which may be two to six months and may involve the patient's symptoms getting worse before they get better) so a short-term trial stimulation does not mean longer-term stimulation will not be successful. The length of time to achieve therapeutic effect means that the mechanism of impact involves neural plasticity. Also that anterior-pain symptoms decrease as well as posterior-pain symptoms indicates that a central mechanism is involved. In addition, for hemicrania continua, pain remediation may be separate from autonomic symptoms such as rhinorrhea and tearing excess that can remain after headache symptoms decrease. Meningeal and Greater Occipital Nerve inputs come together, not peripherally but centrally at the second-order neuron in the spinal cord (Bartsch, T. and P. J. Goadsby, “Stimulation of the greater occipital nerve induces increased central excitability of the dural afferent input,” Brain, 125:1496-1509, 2002.) indicating involvement of the caudal trigeminal nucleus and the upper cervical segments and suggesting a mechanism for referred pain.

A suggested mechanism for the etiology of headache is sensitization of the brainstem because of the sensory input from the occipital nerve causing altered neural processing (Muehlberger, T., Brittner, W., Buschmann, A., and T. Nidal Toman, “Lasting Outcome of the Surgical Treatment of Migraine Headaches—a Four Year Follow-up,” Abstract #14728, Meeting of the American Society of Plastic Surgery, Nov. 3, 2008).

For the treatment of migraine and cluster headaches and other conditions, it would be of benefit to apply a non-invasive treatment modality.

### SUMMARY OF THE INVENTION

It is the purpose of this invention to provide methods and systems and methods for ultrasound neuromodulation of the occipital nerves. Such neuromodulation can effectively used for the treatment of migraine and cluster headaches in their multiple variations as well other pain, tension, and other conditions.

### BRIEF DESCRIPTION OF THE DRAWINGS

FIG. 1 shows an ultrasound transducer against the occiput of the patient targeting the occipital nerves using an embodiment using a either a unilateral ultrasound transducer or a pair of ultrasound transducers for bilateral stimulation.

FIG. 2 shows a diagram of the occipital nerves relative to the occiput.

FIG. 3 shows a block diagram of the control circuit.

### DETAILED DESCRIPTION OF THE INVENTION

It is the purpose of this invention to provide methods and systems and methods for ultrasound neuromodulation of the occipital nerves. The neuromodulation of the occipital nerves in turn neuromodulates connected intracranial structures to obtain therapeutic results.

The acoustic frequency (e.g., typically in that range of 0.3 MHz to 0.8 MHz whether cranial bone is to be penetrated or not) is gated at the lower rate to impact the neuronal structures as desired. A rate of 300 Hz (or lower) causes inhibition (down-regulation) (depending on condition and patient). A rate in the range of 500 Hz to 5 MHz causes excitation (up-regulation)). Power is generally applied at a level less than 60 mW/cm2. Ultrasound pulses may be monophasic or biphasic, the choice made based on the specific patient and condition. Ultrasound stimulators are well known and widely available.

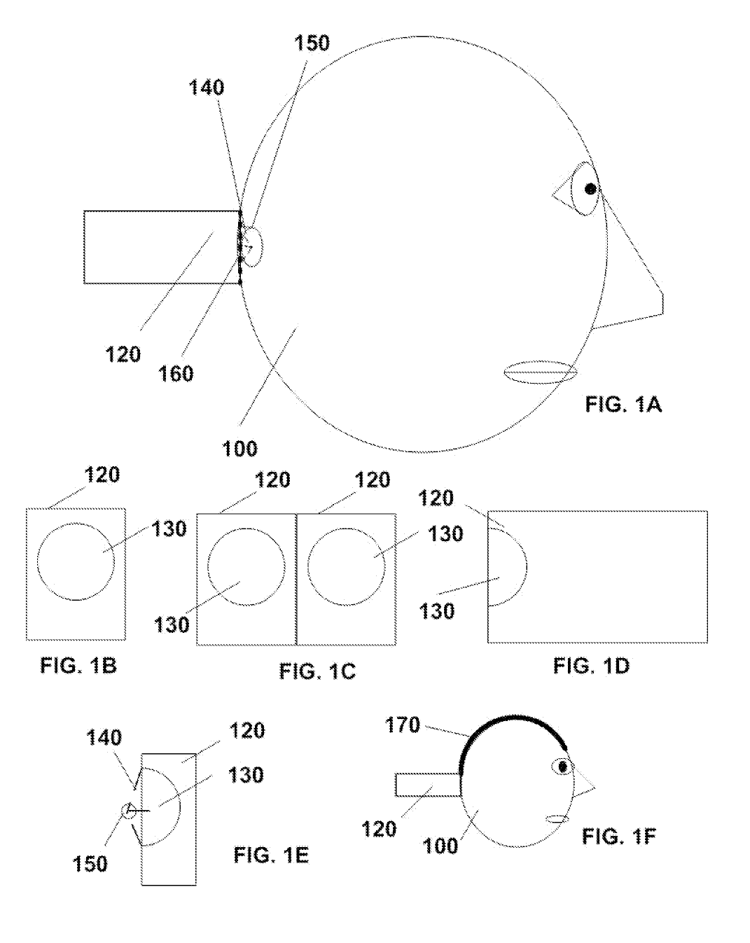

FIG. 1A shows a saggital view of the configuration for neuromodulation of the occipital nerve. Patient head 100 contains occipital nerve bundle 150. Ultrasound transducer 120 focuses sound field 140 on occipital nerve bundle 150. For the ultrasound to be effectively transmitted through intervening tissue to the neural targets, coupling must be put into place. Ultrasound transmission medium (e.g., Dermasol from California Medical Innovations or silicone oil in a containment pouch) is used as insert within the ultrasonic transducer (130 in FIGS. 1B-1E). Ultrasound gel layer 160 that provides the interface for ultrasound conduction between ultrasound transducer 120 and head 100 completes the conduction pathway.

Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length with a suitable lens and/or ultrasound conduction medium. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well. Ultrasound conduction medium will be required to fill the space. If patient sees impact, he or she can move transducer in the X-Y direction (Z direction is along the length of transducer holder and could be adjusted as well). The elongated shape is convenient for the patient to hold and also for use with a positioning headband as shown in FIG. 1F showing patient head 100 with ultrasound transducer 120 and anterior-posterior headband 170. A hat style or open frame with side-to-side stabilization (neither shown) can be employed as alternative embodiments. Ultrasound transducer 120 is moved in and out of a holder (not shown) to provide the appropriate distance between ultrasonic transducer 120 and occipital nerve bundle target 150. In other embodiments, alternative fixed configurations, either of different ultrasonic transducer focal lengths or of different fixed positions in holders, are available for selection for specific patients.

As to X-Y position on the head, the treatment for a specific patient can be planned using physical landmarks on the patient. Loukas et al. (Loukas, M., El-Sedfy, A., Tubbs, R. S., Louis Jr., R. G., Wartmann, Ch. T., Curry, B., and R. Jordan, “Identification of greater occipital nerve landmarks for the treatment of occipital neuralgia,” Folia Morphol., Vol. 65, No. 4, pp. 337-342, 2006) used an approach that takes patient skull size into account. While the location of the Greater Occipital Nerve for anesthesia or any other neurosurgical procedure is typically viewed as “one thumb's breadth lateral to the external occipital protuberance (2 cm laterally) and approximately at the base of the thumb nail (2 cm inferior),” the study found the appropriate point was located “approximately 41% of the distance along the inter-mastoid line (medial to a mastoid process) and 22% of the distance between the external occipital protuberance and the mastoid process.” In addition, the patient can adjust positioning based on effect.

Ultrasound transducer 120 with ultrasound-conduction-medium insert 130 are shown in front view in FIG. 1B for a single transducer 120 for unilateral and in FIG. 1C for pair of transducers 120 for bilateral stimulation. A side view of the same elements in shown in FIG. 1D. FIG. 1E again shows a side view of ultrasound transducer 120 and ultrasound-conduction-medium insert 130 with ultrasound field 140 focused on the occipital nerve bundle target 150. The focus of ultrasound transducer 120 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Transducer arrays of the type 120 may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. The design of the individual array elements and power applied will determine whether the ultrasound is high intensity or low intensity (or medium intensity) and because the ultrasound transducers are custom, any mechanical or electrical changes can be made, if and as required. Vendors such as Blatek and Keramos-Etalon in the U.S. and Imasonic in France can supply suitable ultrasound transducers.

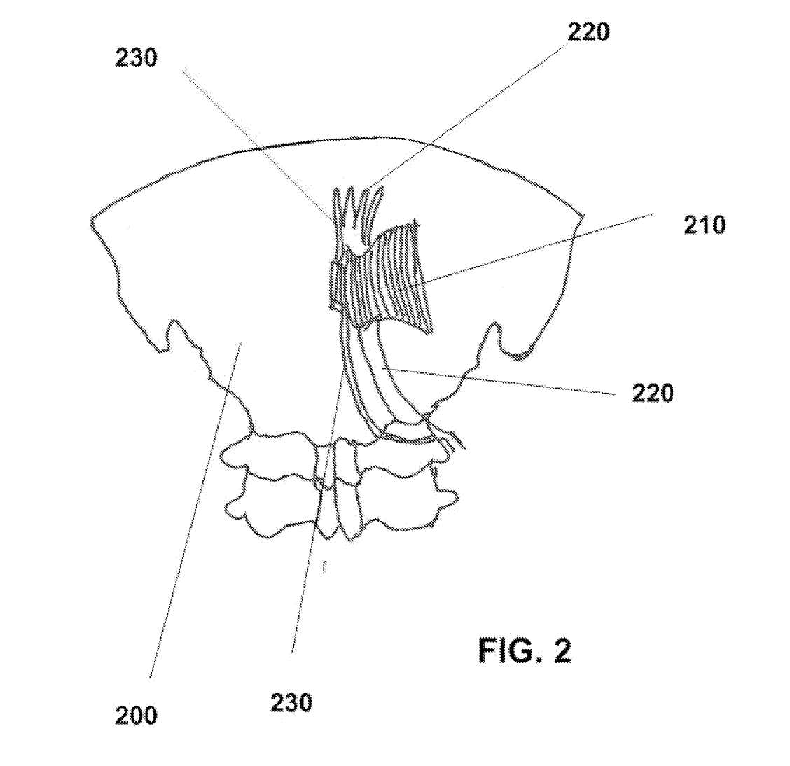

FIG. 2 anatomy of the occiput illustrating the location of occipital nerves. Occipital bone section 200 has trapezius muscle complex 210 through which the Greater Occipital Nerve 220 and the Third Occipital Nerve 230 pass. The occipital nerves occur bilaterally. Neuromodulation of which side will be most effective is headache specific and patient specific. In an alternative embodiment, bilateral neuromodulation will be supplied and this will be the usual situation. In another embodiment, the current invention will be applied to one side of the patient and an alternative treatment to the other side. Alternative invasive treatments have been electrical stimulation, local anesthetic blocks, surgical transection, surgical resection, radiofrequency, alcohol/phenol infiltration, radiosurgery, and cryotherapy. Medications and other non-invasive treatments such as avoidance of triggers, diet modification, physical therapy, chiropractic manipulation, and acupuncture have been used as well.

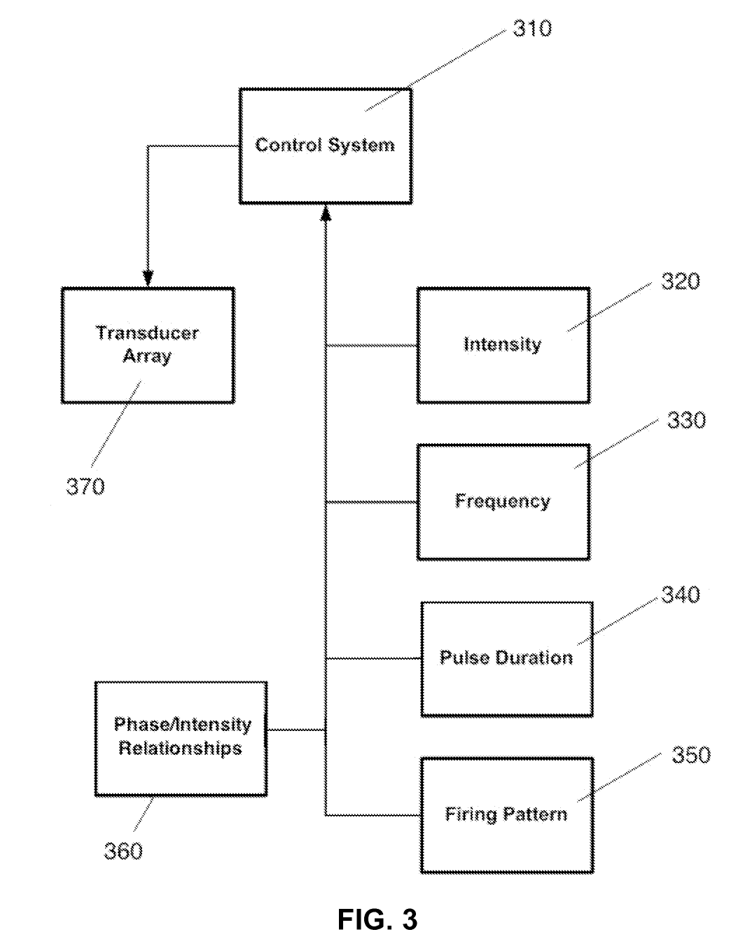

FIG. 3 illustrates the control circuit. Control System 310 receives its input from Intensity setting 320, Frequency setting 330, Pulse-Duration setting 340, Firing-Pattern setting 350, and Phase/Intensity Relationships 360. Control System 310 then provides output to drive Transducer Array 370 and thus deliver the neuromodulation. Settings may be input by the healthcare professional or, under the prescription and directions of a physician, set by the patient.

As indicated by previous work noted above for electrical stimulation, the positive effect of treatment, so that in addition to any acute positive effect, there will be a long-term “training effect” with Long-Term Depression (LTP) and Long-Term Potentiation (LTD) depending on the central intracranial targets to which the occipital nerve is connected.

The invention can be applied to a number of conditions including headaches in various forms, migraine headaches in various forms, cluster headaches in various forms, neuralgias, facial, and other pain or tension syndromes.

Kovacs et al. (Kovacs, S. Peeters, R., De Ridder, D., Plazier, M., Menovsky, T. and S. Sunaert, “Central Effects of Occipital Nerve Electrical Stimulation Studied by Functional Resonance Imaging,” Neuromodulation: Technology at the Neural Interface, Vol 14, Issue 1, pages 46-57, January/February 2011, Article first published online: 7 DEC 2010 DOI: 10.1111/j.1525-1403.2010.00312.x) applied electrical stimulation of the occipital nerve and looked at the impact on neural structures as determined through fMRI. As shown in the fMRI, major areas of activation were the hypothalami, the thalami, the orbito-frontal cortex, the prefrontal cortex, periaqueductal gray, the inferior parietal lobe, and the cerebellum. As to deactivation, the major areas were in the primary motor area (M1) the primary visual area (V1), the primary auditory area (A1), and the somatosensory (S1), the amygdala, the paracentral lobule, the hippocampus, the secondary somatosensory area (S2), and the supplementary motor area (SMA). Ultrasound neuromodulation provided by the current invention would have activate and deactivate the same structures and thus can provide therapeutic effects related to the neuromodulation of those structures.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What I claim is:

1. A method of non-invasively neuromodulating the target occipital nerves using ultrasound stimulation, the method comprising:
aiming an ultrasound transducer at the target,
applying pulsed power to said ultrasound transducer via a control circuit thereby modulating the activity of the target,
whereby therapeutic results are obtained.

2. The method of claim 1, wherein the plurality of control elements is selected from the group consisting of intensity, frequency, pulse duration, firing pattern, and phase/intensity relationships.

3. The method of claim 1 focusing the sound field of an ultrasound transducer at the target occipital nerves neuromodulating the activity of the target in a manner selected from the group of up regulation and down regulation.

4. The method of claim 1, wherein the acoustic ultrasound frequency is in the range of 0.3 MHz to 0.8 MHz.

5. The method of claim 1, where in the power applied is less than 60 mW/cm².

6. The method of claim 1, wherein the configuration of ultrasound power is selected from the group consisting of monophasic and biphasic.

7. The method of claim 1, wherein a stimulation frequency for of 300 Hz or lower is applied for inhibition of neural activity.

8. The method of claim 1, wherein the stimulation frequency for excitation is in the range of 500 Hz to 5 MHz for excitation of neural activity.

9. The method of claim 1, wherein the focus area of the pulsed ultrasound is 0.1 to 0.6 inches in diameter.

10. The method of claim 1, wherein the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

11. The method of claim 1, wherein the neuromodulation of the occipital nerves is selected from the group consisting of unilateral and bilateral.

12. The method of claim 1, wherein the neuromodulation results in a durable effect selected from the group consisting of Long-Term Potentiation and Long-Term Depression.

13. The method of claim 1, wherein the disorder treated is selected from the group consisting of headaches in various forms, migraine headaches in various forms, cluster headaches in various forms, neuralgias, facial and other pain or tension syndromes.

14. The method of claim 1, wherein the ultrasound neuromodulation results in activation of the hypothalami, the thalami, the orbito-frontal cortex, the prefrontal cortex, periaqueductal gray, the inferior parietal lobe, and the cerebellum.

15. The method of claim 1, wherein the ultrasound neuromodulation results in deactivation of the primary motor area (M1) the primary visual area (V1), the primary auditory area (A1), and the somatosensory (S1), the amygdala, the paracentral lobule, the hippocampus, the secondary somatosensory area (S2), and the supplementary motor area (SMA).

16. The method of claim 1, wherein ultrasound therapy is combined with one or more therapies selected from the group consisting of medications, electrical stimulation, local anesthetic blocks, surgical transection, surgical resection, radiofrequency, alcohol/phenol infiltration, radiosurgery, cryotherapy, medication, avoidance of triggers, diet modification, physical therapy, chiropractic manipulation, and acupuncture.
